(19) United States
(12) Patent Application Publication
(10) Pub. No.: US 2025/0250595 A1
(43) Pub. Date: Aug. 7, 2025

(54) PROCESS FOR PRODUCTION OF HEME IRON

(71) Applicant: COLORADO BIOLABS, INC., Lafayette, CO (US)

(72) Inventors: Adam Kashenberg, Englewood, CO (US); Justin Loftin, Cozad, NE (US)

(21) Appl. No.: 19/046,773

(22) Filed: Feb. 6, 2025

Related U.S. Application Data
(60) Provisional application No. 63/550,370, filed on Feb. 6, 2024.

(51) Int. Cl. C12P 13/00

(52) U.S. Cl. CPC C12P 13/00

(57) ABSTRACT

A heme iron production process is disclosed. This process includes operations of separating red blood cells from whole blood via centrifuge, collecting the red blood cells, transferring the red blood cells into high speed centrifuges, centrifuging at a speed greater than 6000 rpm, combining effluent red blood cells with reverse osmosis water, increasing temperature and pH of the water, enzymatically digesting the effluent red blood cells into heme iron molecules and waste, and separating the waste from the heme iron molecules.

### CROSS REFERENCE TO RELATED APPLICATIONS

This application claims the benefit of priority to U.S. Provisional Patent Application Ser. No. 63/550,370 filed Feb. 6, 2024.

### BACKGROUND OF THE DISCLOSURE

The present disclosure relates to production of an iron product and more particular to production of a heme iron molecular concentrate from hemoglobin.

A current heme iron production process is as follows: After a cow has been euthanized and hung the cow's throat is cut and the blood is drained into a bucket. Each cow gets its own bucket. After the cow is fully drained, the bucket is transferred to a separate chain system to be transported to a blood room. Once it reaches the blood room, the blood bucket, is dumped into a collection tank and the blood is pumped from there into a centrifuge. This separates red blood cells from Plasma consisting of all other components of the raw blood. The red blood cells are pumped from the centrifuge to one on two locations: either a dryer to be dried into blood mill or a chiller to be chilled and put into large plastic containers called “totes” for further processing.

Once the totes are filled with red blood cells, or RBCs, they are transported to a further processing facility for use within a predetermined period, typically 14 days. The totes full of RBCs are stored in a climate-controlled refrigeration unit just before processing. Next the totes are staged for processing, and the RBCs in the totes are pumped into tanks with a predetermined amount of RO water (reverse osmosis water).

Once the RBC's start mixing with the RO water this starts a process called Hemolysis. This is the breaking of the red blood cell walls by osmosis. The red blood cell will take on so much RO water that it will burst open. Once all the blood is pumped into the tank the temperature and pH of the blood and water solution is brought up to a specification to start the next process, called Denaturation. Denaturation is given a set amount of time for the hemoglobin molecule to open. Before this process starts the iron in the hemoglobin is wrapped by a tightly wound bundle of protein strands. Denaturation gets the protein strands to open and unbundle, so we can get to the iron within. Once this process has set for a while, all the while keeping an eye on pH and temperature. An Enzyme is added to break down the protein strands surrounding the iron. This starts the digestion phase of the process. Digestion is given a minimum of two hours for the enzyme to break down the proteins.

During this digestion process there is also a water addition to help keep the hemoglobin suspended in the solution. After the minimum digestion time the tanks are connected to filters to filter out any of the digested materials. Filtration will continue until the process is concentrated down and the effluent from the filters hits a predetermined output.

Once the filtration is concluded the remaining digest is pumped into a cooker to start the cooking part of the process. This process has two purposes, to stop enzyme activity and a pasteurization step. This is achieved by heating the digest mixture to a very high temperature for a period that will stop the enzyme activity and kill any microbes present in the batch, if any are present. After cooking the digest is cooled and pumped to a dryer room for drying. The dryer pulls hot air across the digest as it is sprayed in a very fine mist to almost dry the liquid instantly into a powder. The powder is collected and blended then bagged for storage and later use as a completed product.

### DETAILED DESCRIPTION

As in a conventional process, after a cow has been euthanized and hung the cow's throat is cut and the blood is drained into a bucket. Each cow gets its own bucket. After the cow is fully drained, the bucket is transferred to a separate chain system to be transported to a blood room. Once it reaches the blood room, the blood bucket, is dumped into a collection tank and the blood is pumped from there into a centrifuge. This separates red blood cells from Plasma consisting of all other components of the raw blood. The red blood cells are pumped from the centrifuge to one on two locations: the dryer to be dried into blood mill or a chiller to be chilled and put into large plastic containers called “totes” for further processing.

Once the totes are filled with red blood cells, or RBCs, they may be transported to a further processing facility for use within a predetermined period, typically 14 days. In either the same facility or the further processing facility, the totes full of RBCs are stored in a climate-controlled refrigeration unit just before processing. Next the totes are staged for processing, and the RBCs in the totes are pumped into one or more high speed centrifuges that operate at speeds above 6000 rpm. This generates a residual blood material.

Once the high speed centrifuge operation is completed, RO water is added to the residual blood material and the solution of residual blood material and water is transferred to tanks. At this point in the improved process the RBCs should be completely broken down such that there is no need for a Hemolysis step as previously has been required, and may also eliminate the need for the Denaturation step, described again below.

Once all the blood residual material is pumped into the tanks the temperature and pH of the blood and water residual solution is brought up to a predetermined specification or range to start the next process, called Denaturation. Denaturation is a holding operation in the tanks, i.e. given a set amount of time for the hemoglobin molecules in the residual blood material to open. Finally, an Enzyme is added to the solution in the tank to break down the protein strands surrounding the heme iron molecules and begin the digestion phase of the process. Digestion is given a minimum of two hours for the enzyme to break down the proteins.

During this digestion phase of the process RO water is added to help keep the hemoglobin suspended in the solution. After at least the minimum digestion time the tanks are connected to filters to filter out any of the digested materials, e.g. broken protein strands from the hemoglobin. Filtration will continue until the process is concentrated down and the effluent digest mixture from the filters hits a predetermined output concentration.

Once the filtration is concluded the remaining digest is pumped into a cooker to start the cooking part of the process. This process has two purposes, to stop enzyme activity and pasteurize the digest mixture. This is achieved by heating the digest mixture to a very high temperature for a period that will stop the enzyme activity and kill any microbes present in the batch, if any are present. After cooking the digest is cooled and pumped to a dryer room for drying. The dryer pulls hot air across the digest as it is sprayed in a very fine mist to almost dry the liquid instantly into a powder. The powder is collected and blended then bagged for storage and later use as a completed product.

The addition of a high speed centrifuge separation step before introduction of RO water essentially replaces at least the hemolysis step and may also eliminate the need for the above described denaturation step currently required in the heme iron preparation process. Accordingly, all such alternatives, variations and modifications are intended to be encompassed within the scope of and as defined by the following claims.

What is claimed is:

1. A heme iron production process comprising steps of:
separation of red blood cells from whole blood by a first centrifuge operation into red blood cells and plasma;
collecting the red blood cells into one or more containers;
transferring the red blood cells into one or more high speed centrifuges;
centrifuging the red blood cells in the one or more high speed centrifuges at a speed greater than 6000 rpm to generate effluent red blood cells;
combining the effluent red blood cells with reverse osmosis water;
increasing temperature and pH of the effluent red blood cells and RO water;
adding an enzyme to digest the effluent red blood cells into a waste product and heme iron molecules; and
separating the waste product from the heme iron molecules.
